(19) United States
(12) Patent Application Publication
(10) Pub. No.: US 2017/0340658 A1
(43) Pub. Date: Nov. 30, 2017

(54) COMBINED USE OF A CHEMOTHERAPEUTIC AGENT AND A CYCLIC DINUCLEOTIDE FOR CANCER TREATMENT

(71) Applicant: INVIVOGEN, Toulouse (FR)

(72) Inventors: Fabienne VERNEJOUL, Toulouse (FR); Daniel DROCOURT, Saint Orens De Gameville (FR); Jesus ROMO, California, CA (US); Gerard TIRABY, Toulouse (FR); Thierry LIOUX, Balma (FR)

(21) Appl. No.: 15/535,864

(22) PCT Filed: Dec. 9, 2015
(86) PCT No.: PCT/EP2015/079171

§ 371 Date: Jun. 14, 2017

(30) Foreign Application Priority Data
Dec. 16, 2014 (EP) 14307058.9

(51) Int. Cl. A61K 31/7068; A61K 31/7084

(52) U.S. Cl. CPC A61K 31/7068; A61K 31/7084; A61K 2300/00

(57) ABSTRACT

A kit of parts includes a) gemcitabine or a pharmaceutically acceptable salt thereof and b) a cyclic dinucleotide or pharmaceutically acceptable salt thereof, wherein the cyclic dinucleotide or pharmaceutically acceptable salt thereof is an agonist of the receptor known as “stimulator of interferon genes” (STING), for use in the treatment of solid pancreatic cancer.

### FIELD OF THE INVENTION

The present invention relates to the combination of a chemotherapeutic agent with a cyclic dinucleotide for use in the treatment of cancer, particularly of solid pancreatic tumor. The present invention further relates to specific cyclic dinucleotides useful for treating cancer.

### BACKGROUND OF THE INVENTION

Cancer is a loosely related family of diseases characterized by uncontrolled cell growth and division. Together, the over 200 known forms of cancer inflict a terrible social burden in terms of loss of life, diminished quality of life, healthcare costs and reduced productivity. Although major strides have been made in the diagnosis and treatment of certain cancers over the past few decades, there remains a pressing need for new treatments adapted to each type of cancer and to the specific needs of each patient.

Cancers are usually treated with some combination of surgery, chemotherapy (i.e. drugs), and/or radiation therapy. Surgery is used to resect solid tumors, whereas chemotherapy and radiation therapy, which can be local or systemic, are used to stop the growth of, shrink and/or destroy tumors, and/or to prevent tumors from metastasizing. The primary drawback of most chemotherapeutic agents and radiation treatments is that they fail to distinguish between tumors and healthy tissue. This is because they target the most rapidly dividing cells in the body, which encompass tumor cells as well as healthy cells that normally divide at a fast rate (e.g. germ, hair, or stomach-lining cells). This lack of discrepancy explains the numerous side effects of chemotherapy and radiation therapies, including myelosuppression (reduced production of blood cells), immunosuppression, inflammation, disrupted functionality of the ovaries or testes, hair loss, asthenia (generalized weakness), extreme fatigue, nausea and loss of appetite.

Pancreatic cancer is the fourth leading cause of cancer-related deaths in Europe and in the USA (Malvezzi, Bertuccio, Levi, La Vecchia, & Negri, 2013). The prognosis for pancreatic cancer remains grim: the 1-year survival rate is only 26%, the 5-year survival rate only 6% and the average life expectancy following diagnosis with metastatic disease just 3 to 6 months (Hershberg Foundation, 2014). Thus, despite representing only about 3% of cancers in the USA, pancreatic cancer accounts for roughly 7% of cancer deaths (American Cancer Society, 2014). Despite encouraging progress in the treatment of many other cancers since the late 1980's, pancreatic cancer is the only cancer that shows unfavorable trends for both sexes, and the only one for men (Malvezzi et al., 2013). Modern treatment regimens for pancreatic cancer depend on the cancer type and stage as well as the patient's clinical status, but typically involve surgical resection of the tumor (in Stages 1 and 2), chemotherapy and/or radiation therapy. The most common chemotherapeutic agents for pancreatic cancer are gemcitabine and 5-fluorouracil.

Gemcitabine is a widely used cancer chemotherapeutic that is the standard treatment for non-resectable pancreatic cancer (Shindo et al., 2014). It is the first-line treatment for patients with locally advanced (non-resectable Stage 2 or 3) or metastatic (Stage 4) pancreatic adenocarcinoma. Furthermore, gemcitabine is indicated for certain relapsed ovarian cancers (in combination with carboplatin, as a secondary treatment), some types of metastatic breast cancer (in combination with paclitaxel as a first-line treatment), and some inoperable advanced or metastatic non-small lung cancers (in combination with cisplatin, as a first-line treatment). Gemcitabine is a nucleoside analog that kills tumor cells by blocking DNA replication at multiple steps. Additionally, there is ever-increasing evidence that gemcitabine has other activities. For instance, it has been shown to selectively eliminate myeloid suppressor cells in the spleens of tumor-bearing mice without markedly diminishing beneficial immune cells (e.g. CD4+ T cells, CD8+ T cells, natural killer [NK] cells, macrophages or B cells), an effect that leads to increased anti-tumor activity of CD8+ T cells and NK cells (Suzuki, Kapoor, Jassar, Kaiser, & Albelda, 2005). Despite its efficacy, gemcitabine causes similar side effects to other common chemotherapeutic agents.

Another approach for treating cancer is immunotherapy, which consists of activating the patient's own immune system to fight against the disease. Cancer immunotherapy agents include nucleic acids, cytokines, peptides, proteins, immune cells (endogenous, or conferred with anti-cancer activity ex vivo), fragments of bacteria or viruses, and synthetic drugs. They can be used to elicit a specific immune response against a particular cancer cell type, or to trigger a general immune response that indirectly targets cancer cells or their effects. The former is typically achieved with antibodies or vaccines that target one or more antigens on or in cancer cells. General immunotherapy is usually done with immunomodulatory agents and/or chemical entities that simultaneously activate one or more types of immune cells to fight against cancer cells.

There is some literature precedent on the combined use of gemcitabine and some form of immunotherapy, especially for treatment of pancreatic cancer. Hirooka et al. evaluated a combination therapy comprising gemcitabine and a dendritic cell (DC)-based vaccination in five patients with inoperable, locally advanced pancreatic cancer (Hirooka et al., 2009). The vaccination consisted of intratumoral injection of activated DCs (DCs pulsed with the antineoplastic bacterial agent OK432 [picibanil]), followed by infusion of lymphokine-activated killer (LAK) cells stimulated with anti-CD3 monoclonal antibody. They reported positive results in three of the five patients: one that exhibited partial remission, and two that showed long-term stable disease (>6 months). In the patient with remission, they observed induction of antigen-specific cytotoxic T lymphocytes—and effect that they attributed to the synergic effects between gemcitabine and the DC-based vaccination. In closely related work, Kimura et al. assessed the safety and efficacy of a combination of DC-based immunotherapy (with or without LAK cells) and chemotherapy (either gemcitabine or S-1) in a cohort of 49 patients with inoperable pancreatic carcinoma refractory to standard treatment (Kimura et al., 2012). The authors state that prolongation of survival in the cohort was “highly likely”. They explain that the patients that had received DC vaccine and chemotherapy plus LAK cells survived longer than did those who had received the analogous treatment without LAK cells, and associated the longer survival with the decreased number of regulatory T cells observed in several of the patients. Using another approach, Nishida et al. recently completed a Phase I study on a combination of Wills tumor gene (WT-1) peptide-based vaccine, and gemcitabine, in a cohort of 32 patients with advanced pancreatic cancer (Nishida et al., 2014). They reported that the treatment was well tolerated in the patients and they preliminarily affirmed that it “seemed to be better than that of gemcitabine alone”, especially in terms of survival. They have since begun a Phase II randomized clinical trial to further ascertain its efficacy.

Preliminary studies on combinations of gemcitabine and either a cytokine or derivative thereof, such as IFN-α (Fritz, 2015) (US Patent Application 2014/219961 A1), IFN-β (Tomimaru, 2014) or TNF-α (Murugesan, 2009), all suggest that such combinations might provide advantages over gemcitabine monotherapy for different types of cancer. However, as direct administration of cytokines to patients is well known to often be toxic, in clinical setting gemcitabine could be combined with some immunomodulatory substance that induces cytokines once in the body, perhaps only locally, where they are needed. Furthermore, U.S. Pat. No. 7,851,599 relates to a chemoimmunotherapy that combines an antibody-interleukin-2 (IL-2)-fusion protein with gemcitabine; WO2010014784 A9 refers to the combined use of an anti-CTLA4 antibody and various chemotherapeutic agents, including gemcitabine.

Particularly relevant to the present invention was a Phase I clinical trial that we performed in collaboration with the group of Buscail, in which we demonstrated the safety and efficacy of gemcitabine combined with a proprietary DNA plasmid gene therapy product known as CYL-02, in a cohort of 22 patients with pancreatic ductal adenocarcinoma ((Buscail L., 2015) and EP 2047858 A1). We originally attributed the efficacy of the treatment principally to expression of the genes contained in CYL-02, which encode proteins with known anti-proliferative and anti-metastatic activity. However, upon subsequently testing plasma samples from the original patient cohort, we observed that CYL-02 induced Type I interferons to different levels in many patients. Given these results, the well-established use of IFN-α in clinical oncology, and mounting evidence of the local anti-tumor effects of IFN-α, we can now explain the efficacy of CYL-02 according to two complimentary mechanisms: expression of the aforementioned genes and induction of therapeutically beneficial cytokines, the latter of which would occur via stimulator of interferon genes (STING)-regulated, DNA-mediated induction of Type I interferons (Ishikawa, Ma, & Barber, 2009) (Klarquist et al., 2014).

A major player in physiological production of cytokines is STING (also known as ERIS, MITA, MPYS, or TM173), a transmembrane receptor protein that is paramount in innate immunity. Human STING is encoded by the gene TMEM173. Activation of STING leads to production of Type I interferons (e.g. IFN-α and IFN-β), via the IRF3 (interferon regulatory factor 3) pathway; and to production of pro-inflammatory cytokines (e.g. TNF-α and IL-113), via the NF-κB pathway and/or the NLRP3 inflammasome (Abdul-Sater et al., 2013). A recent report described an unusual activity of gemcitabine: its ability to prevent inhibition of STING (Mitzel, 2014). Specifically, the authors found that in macrophages and in mouse models of viral infection, gemcitabine treatment led to greater STING-dependent production of IFN-β, by reducing inhibition of STING by the protein Atg9A.

Human and murine STING are naturally activated two ways: via binding of exogenous (3′,3) cyclic dinucleotides (c-diGMP, c-diAMP and c-GAMP) that are released by invading bacteria or archaea (see (Gomelsky, 2011) and references therein); and via binding of (2′,3′)cyclic guanosine monophosphate-adenosine monophosphate ((2′,3′)c-GAMP), a recently discovered endogenous cyclic dinucleotide that is produced by the enzyme cyclic GMP-AMP synthase (cGAS; also known as C6orf150 or MB21D1) in the presence of exogenous double-stranded DNA (e.g. that released by invading bacteria, viruses or protozoa) or of self-DNA in mammals (see, for example: (Ablasser et al., 2013) and (Zhang et al., 2013)). Moreover, synthetic analogs of the aforementioned naturally occurring cyclic dinucleotides can activate the STING pathway (see, for example: (Dubensky, Kanne, & Leong, 2013) and (Li et al., 2014))

Some cyclic dinucleotides have been described as having immunomodulatory properties that could be exploited in an immunotherapy treatment. This immunomodulatory activity is typically demonstrated by showing that these compounds induce cytokines and/or activate immune cells in vitro or in vivo. The related U.S. Pat. Nos. 7,569,555 B2 and 7,592,326 B2 refer to administration of c-diGMP or functionally equivalent analogs thereof as a “method of stimulating and/or modulating the immune and inflammatory response”. They suggest that these compounds could be used to prevent or treat allergic reactions, or as vaccine adjuvants. They demonstrate that c-diGMP induces diverse cytokines, including chemokines, in cell lines in vitro, and can be used together with an antigen to activate dendritic cells in vitro. US patent application 2008/0286296 A1 refers to the use of c-diGMP, c-diAMP and 3′,3′ cyclic dinucleotide analogs thereof as “adjuvants or and/or immunomodulators for prophylactic and/or therapeutic vaccination” for a wide range of indications. The authors reported that c-diGMP stimulates murine DC cells to produce CD40 in vitro. Moreover, in diverse experiments on murine models of immunization (using β-galactosidase as antigen), the authors show that mice treated with c-diGMP or c-diAMP post-immunization produce greater amounts of various cytokines, and/or IgG, and/or anti-β-Gal antibodies than do mice that do not receive any cyclic dinucleotide. US patent application 2014/0205653 A1 and the related WIPO patent application 2014/093936 A1 encompass the synthesis, and immunomodulation activity screening, of stereochemically-defined 3′,3′ cyclic dinucleotides, including phosphorothioate (also known as “P(S)” or “thiophosphate”) analogs. They report that representative compounds of their invention induce IFN-β in vitro in two cell lines: THP-1 human monocytes and DC2.4 cells. Furthermore, they describe the efficacy of some of these compounds in murine models of immunization in which SIV gag protein or OVA were used as antigen. Specifically, they report that SIV-gag-immunized mice treated with (Rp,Rp)dithio-diphosphate c-diGMP exhibit better SIV-gag-specific CD8 T cell memory than do controls treated with saline, and that OVA-immunized mice treated with (Rp,Rp)dithio-diphosphate c-diGMP exhibit better OVA-specific CD8 T cell memory than do those treated with the reference compound c-diGMP.

In 2006, Romling (Romling & Amikam, 2006) suggested that the effects of c-diGMP in eukaryotes might be exploited for cancer treatment, while the group of Karaolis reported that c-diGMP inhibited the growth of human colon cancer (H508) cells in vitro, suggesting that cyclic dinucleotides could be used as therapeutic agents for cancer treatment or prevention (Karaolis, 2005; U.S. Pat. No. 7,709,458 B2).

Dubensky and colleagues have published an extensive review of STING agonist cyclic dinucleotides used as adjuvants, outlining work by their group and those of Karaolis, Guzman, and Yan & Chen. Depending on the experiment cited, all the disclosed compounds (c-diGMP, c-diAMP, c-diIMP and related analogs, including 2′,3′ and 3′,3′ compounds) induced production of various cytokines (e.g. Type I interferons, TNF-α, IL-2, etc.) either in vitro or in vivo (in healthy animals or in animal models of disease) (Dubensky et al., 2013). The type and extent of immunomodulation by cyclic dinucleotides is partially dictated by the cells on which they act.

Recently, Miyabe et al. (Miyabe et al., 2014) demonstrated the efficacy of a combination therapy of c-diGMP plus OVA in mice that received different immunization treatments followed by subcutaneous injection of E.G7-OVA tumors. Mice that had been immunized with a combination of c-diGMP, OVA and liposomal carrier showed drastically and significantly smaller tumor volumes than did mice treated with PBS alone, OVA alone, OVA plus c-diGMP, or OVA plus the liposomal carrier. The authors attributed the efficacy of the combination therapy to induction of IFN-β by c-diGMP through the STING-TBK1-IRF3 pathway. Interestingly, Chandra et al. (Chandra et al., 2014) have reported that when mice with breast cancer metastases were immunized with a Listeria monocytogenes (LM)-based vaccine and subsequently treated with the STING agonist c-diGMP, the metastases almost completely disappeared. Ohkuri and colleagues studied the activity of Type I IFNs in the microenvironment of glioma, finding that STING is partially responsible for local production of these cytokines (Ohkuri et al., 2014). They then tested c-diGMP immunotherapy as primary treatment in a murine model of glioma, reporting that mice that had received c-diGMP by intra-tumoral injection exhibited longer survival, more of certain therapeutically beneficial T cells (CD4+ and CD8+ and CD11c+), and greater expression of certain cytokine genes (including CC15 and Cxcl10) than did mice that had received only solvent (Ohkuri et al., 2014). They also showed that c-diGMP inhibited tumor growth in a murine model of de novo glioma. The authors affirmed that under these conditions, c-diGMP enhances recruitment of T cells to the tumor site. Finally, they evaluated c-diGMP as an adjuvant for antigen-specific vaccination of glioma in a murine model of glioma that expresses OVA257-264 as tumor antigen. They reported that although c-diGMP monotherapy provided longer survival than did vaccine alone or negative control (using mock treatment), the longest survival was observed in mice treated with a combination of c-diGMP and anti-OVA257-264 vaccine. In both the primary treatment and the adjuvant studies, the authors observed beneficial effects of c-diGMP-treatment in brain-infiltrating leukocytes (BILs) obtained from each type of treated mouse.

There are very few literature reports of combination therapies that entail use of cyclic dinucleotides. The related patent applications US 2014/0205653 A1 and WO 2013/185052 A1 report the use of cyclic dinucleotide STING agonists, including prodrugs thereof, in combination with the cancer vaccine GVAX (inactivated tumor cells stimulated to release the cytokine GCSF). The authors demonstrate that a combination therapy comprising use of Rp, Rp dithio c-diAMP and GVAX provides greater inhibition of tumor growth in a murine model of TRAMP-C2 subcutaneous tumors than do GVAX monotherapy or the combination of c-diAMP and GVAX.

We have found that the present invention, a specific combination of the chemotherapeutic agent gemcitabine with a ligand of both human and murine STING, which we chose from a panel of synthetic cyclic dinucleotides based on adenosine and inosine, might represent a promising new chemoimmunotherapy for cancer, especially for treating solid pancreatic tumors.

### SUMMARY OF THE INVENTION

The object of the present invention is a kit of parts comprising a chemotherapeutic agent and a stimulator of interferon genes (STING) agonist cyclic dinucleotide or a pharmaceutically acceptable salt or prodrug thereof for use in the treatment of cancer.

In another embodiment, the present invention discloses a method for treating cancer, said method comprising administering to a patient in need thereof:
gemcitabine or a pharmaceutically acceptable salt or prodrug thereof; and
a cyclic dinucleotide or a pharmaceutically acceptable salt or prodrug thereof;
   wherein said cyclic dinucleotide or pharmaceutically acceptable salt or prodrug thereof is a STING agonist.

In one embodiment, the cancer is pancreatic cancer, particularly solid pancreatic tumor.

In a further embodiment, the chemotherapeutic agent is gemcitabine.

Thus in one particular embodiment the present invention relates to a kit of parts comprising:
gemcitabine or a pharmaceutically acceptable salt or prodrug thereof; and
a cyclic dinucleotide or a pharmaceutically acceptable salt or prodrug thereof, wherein said cyclic dinucleotide or pharmaceutically acceptable salt or prodrug thereof is a STING agonist,
   for use in the treatment of solid pancreatic tumors.

In one embodiment, one nucleoside of said cyclic dinucleotide is adenosine (or an analog thereof) and the other nucleoside is inosine (or an analog thereof).

### DETAILED DESCRIPTION OF THE INVENTION

The present invention provides a novel efficient chemoimmunotherapy for treating cancer. The chemoimmunotherapy according to the invention consists in a combination of a chemotherapeutic agent with a STING agonist cyclic dinucleotide or a pharmaceutically acceptable salt or prodrug thereof.

In a first embodiment, the present invention provides a kit of parts comprising:
a chemotherapeutic agent; and
a cyclic dinucleotide or a pharmaceutically acceptable salt or prodrug thereof, wherein said cyclic dinucleotide or a pharmaceutically acceptable salt or prodrug thereof is a STING agonist,
   for use in the treatment of cancer.

In another embodiment, the present invention discloses a method for treating cancer, said method comprising administering to a patient in need thereof:
a chemotherapeutic agent; and
a cyclic dinucleotide or a pharmaceutically acceptable salt or prodrug thereof;
   wherein said cyclic dinucleotide or pharmaceutically acceptable salt or prodrug thereof is a STING agonist.

In a further embodiment, the present invention provides a cyclic dinucleotide or a pharmaceutically acceptable salt or prodrug thereof for use in the treatment of cancer, wherein said cyclic dinucleotide or a pharmaceutically acceptable salt or prodrug thereof is a STING agonist.

The term “kit-of-parts” herein refers to a combined preparation wherein the active ingredients are physically separated for use in a combined therapy by simultaneous administration or sequential administration to the patient.

Hence, according to the present invention, the chemotherapeutic agent and the cyclic dinucleotide or a pharmaceutically acceptable salt or prodrug thereof are administered to the patient in a separate form, either simultaneously, separately or sequentially in any order, for the treatment of cancer.

The term “cancer” herein refers to the physiological condition in subjects that is characterized by unregulated or dysregulated cell growth or death. The term “cancer” includes solid tumors and blood born tumors, whether malignant or benign.

In a preferred embodiment, the cancer is a cancer from the following group: bladder cancer, breast cancer, cholangiocellular cancer, leukemia, lung cancer, lymphoma, nasopharyngeal cancer, ovarian cancer, pancreatic cancer and urothelial cancer.

The terms “Subject” and “Patient” refer to a human or an animal suffering from cancer.

“Immunotherapy” refers to any medical treatment in which one or more components of a human's or animal's immune system is deliberately modulated in order to directly or indirectly achieve some therapeutic benefit, including systemic and/or local effects, and preventive and/or curative effects.

The term “chemotherapy” herein refers to a medical treatment for cancer with one or more chemotherapeutic agents.

The term “chemotherapeutic agent” herein refers to one or more chemical substances that are administered to a human or animal in order to kill tumors, or slow or stop the growth of tumors, and/or slow or stop the division of cancerous cells and/or prevent or slow metastasis. The chemotherapeutic agent according to the present invention is selected from the following group and includes pharmaceutically acceptable derivatives, salts and prodrugs of each of the following chemotherapeutic agents: gemcitabine, 5-fluorouracil, doxorubicin, paclitaxel and platinum derivatives.

In one further embodiment, the chemotherapeutic agent is gemcitabine.

“Gemcitabine” is a chemotherapeutic agent used in first line treatment of several cancers and is represented by the following formula:

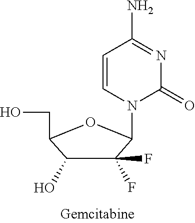

The term “chemoimmunotherapy” herein refers to a combined use, whether sequentially in any order or concurrently, of chemotherapy substances and/or strategies, and immunotherapy substances and/or strategies.

In the present invention, the terms “cyclic dinucleotide” and “CDN” refer to a class of cyclic molecules with two phosphodiester linkages, or two phosphorothioate diester linkages, between two nucleotides. This includes (3′,5′)-(3′,5′) nucleotide linkages (abbreviated as (3′,3′)); (3′,5′)-(2′,5′) nucleotide linkages (abbreviated as (3′,2′)); (2′,5′)-(3′,5′) nucleotide linkages (abbreviated as (2′,3′)); and (2′,5′)-(2′,5′) nucleotide linkages (abbreviated as (2′,2′)).

The term “nucleoside” refers to a glycosylamine constituted of a nitrogenous base and a five-carbon sugar, wherein the nitrogenous base is bound to the five-carbon sugar via a beta-glycosidic linkage.

In a preferred embodiment, the nitrogenous base is a purine derivative.

The term “nucleotide” refers to any nucleoside linked to a phosphate group at the 5′, 3′ or 2′ position of the sugar moiety.

“Pharmaceutically acceptable salts” include those derived from pharmaceutically acceptable inorganic or organic bases and acids. Suitable salts include those derived from alkali metals such as potassium and sodium, alkaline earth metals such as calcium and magnesium, among numerous other acids well known in the pharmaceutical art.

The term “pharmaceutically acceptable prodrug” herein refers to a compound that is metabolized, for example hydrolyzed or oxidized, in the host (i.e. the human or animal subject that receives the compound) to form the compound of the present invention. Typical examples of prodrugs include compounds that have biologically labile protecting groups on functional moieties of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated or dephosphorylated to produce the active compound.

For a comprehensive review, examples of contemplated prodrug forms are described in “Prodrugs” by Kenneth B. Sloan (Sloan, 1992), “Design of Prodrugs” by Hans Bundgaard (Bundgaard, 1985).

“STING” is an abbreviation of “stimulator of interferon genes”, which is also known as “endoplasmic reticulum interferon stimulator (ERIS)”, “mediator of IRF3 activation (MITA)”, “MPYS” or “transmembrane protein 173 (TM173)”. STING is a transmembrane receptor protein and is encoded by the gene TMEM173 in human. In response to viral infection, STING activates STAT6 (signal transducer and activator of transcription 6) to induce (Th2-type), increase (IL-12) or decrease (IL-10) production of various cytokines, including the chemokines CCL2, CCL20, and CCL26 (Chen et al., 2011).

The term “STING agonist” herein refers to a substance that activates the receptor STING in vitro or in vivo. According to the invention, a compound is deemed to be a STING agonist if:
it induces Type I interferons in vitro in human or animal cells that contain STING; and
it does not induce Type I interferons in vitro in human or animal cells that do not contain STING.

A typical test to ascertain whether a ligand is a STING agonist is to incubate the ligand in a wild-type human or animal cell line and in the corresponding cell line in which the STING coding gene has been genetically inactivated by a few bases or a longer deletion (e.g. a homozygous STING knockout cell line). An agonist of STING will induce Type I interferon in the wild-type cells but will not induce Type I interferon in the cells in which STING is inactivated.

Thus, in a particular embodiment, the present invention provides a kit of parts comprising:
a. gemcitabine or a pharmaceutically acceptable salt or prodrug thereof; and
b. a cyclic dinucleotide or pharmaceutically acceptable salt or prodrug thereof,
wherein said cyclic dinucleotide or pharmaceutically acceptable salt or prodrug thereof is an agonist of stimulator of interferon genes (STING),
   for use in the treatment of solid pancreatic tumors.

In a preferred embodiment, the nitrogenous base of each nucleoside of the cyclic dinucleotide is a purine derivative.

In a preferred embodiment, the nitrogenous base of each nucleoside of the cyclic dinucleotide is a purine that is substituted only in position 6 (“6-substituted purine”).

In a more preferred embodiment, one nucleoside of said cyclic dinucleotide is adenosine (or an analog thereof) and the other nucleoside is inosine (or an analog thereof).

In a preferred embodiment, the linkage between the two nucleosides of the cyclic dinucleotide is a (3′,5′)(3′,5′), a (3′,5′)(2′,5′), a (2′,5′)(3′,5′) or a (2′,5′),(2′,5′) phosphodiester and/or phosphorothioate diester linkage, and/or phosphotriester and/or phosphorothioate triester linkage for prodrugs of cyclic dinucleotides.

In one embodiment, the two nucleosides in the cyclic dinucleotide are linked by two phosphodiester linkages.

In another embodiment, the two nucleosides in the cyclic dinucleotide are linked by two phosphorothioate diester linkages.

Particularly preferred CDN for carrying out the present invention are presented in Table 1.

TABLE 1

| Code | Name | Structure |
|---|---|---|
| CL592 | c-AIMP | 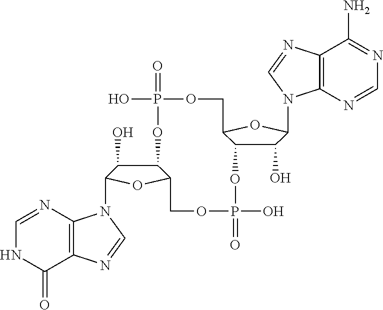 [SMILES: Nc1ncnc2c1ncn2C1OC2COP(=O)(O)O[C@@H]3C(COP(=O)(O)O[C@H]2[C@H]1O)OC(n1cnc2c(=O)[nH]cnc21)[C@@H]3O] |
| CL606 | (3′,2′)c-AIMP | 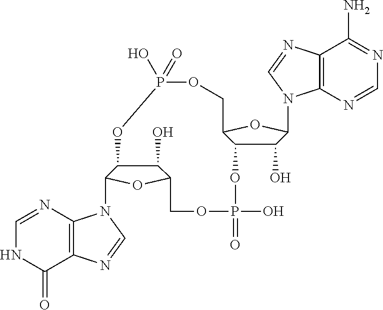 [SMILES: Nc1ncnc2c1ncn2C1OC2COP(=O)(O)O[C@H]3C(n4cnc5c(=O)[nH]cnc54)OC(COP(=O)(O)O[C@H]2[C@H]1O)[C@H]3O] |
| CL611 | (2′,2′)c-AIMP | 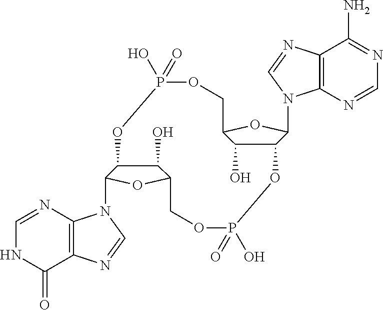 [SMILES: Nc1ncnc2c1ncn2C1OC2COP(=O)(O)O[C@H]3C(n4cnc5c(=O)[nH]cnc54)OC(COP(=O)(O)O[C@@H]1[C@@H]2O)[C@H]3O] |
| CL602 | (2′,3′)c-AIMP | 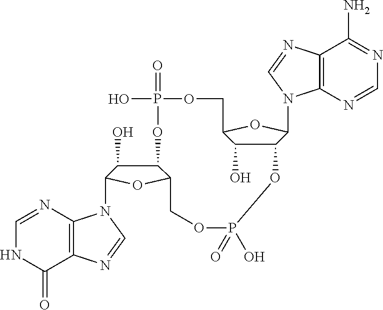 [SMILES: Nc1ncnc2c1ncn2C1OC2COP(=O)(O)O[C@@H]3C(COP(=O)(O)O[C@@H]1[C@@H]2O)OC(n1cnc2c(=O)[nH]cnc21)[C@@H]3O] |
| CL655 | c-AIMP(S) | 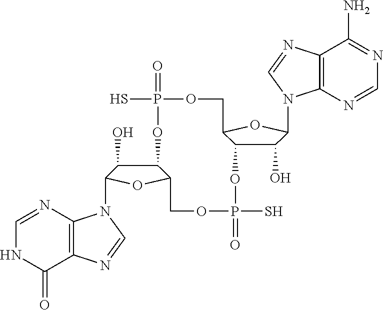 [SMILES: Nc1ncnc2c1ncn2C1OC2COP(=O)(S)O[C@@H]3C(COP(=O)(S)O[C@H]2[C@H]1O)OC(n1cnc2c(=O)[nH]cnc21)[C@@H]3O] |
| CL604 | c-(dAMP-dIMP) | 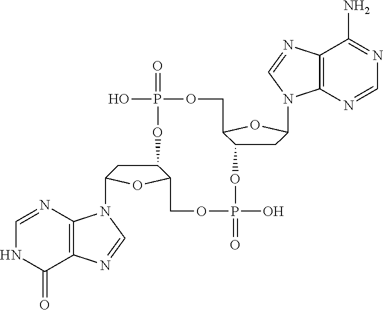 [SMILES: Nc1ncnc2c1ncn2C1C[C@@H]2OP(=O)(O)OCC3OC(n4cnc5c(=O)[nH]cnc54)C[C@@H]3OP(=O)(O)OCC2O1] |
| CL609 | c-(dAMP-2′FdIMP) | 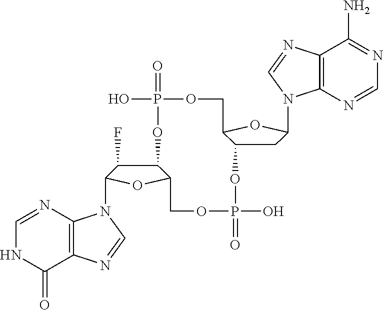 [SMILES: Nc1ncnc2c1ncn2C1C[C@@H]2OP(=O)(O)OCC3OC(n4cnc5c(=O)[nH]cnc54)[C@H](F)[C@@H]3OP(=O)(O)OCC2O1] |
| CL614 | c-(2′FdAMP- 2′FdIMP) | 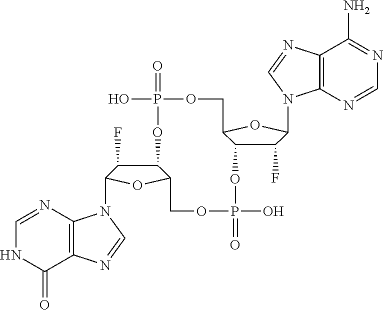 [SMILES: Nc1ncnc2c1ncn2C1OC2COP(=O)(O)O[C@@H]3C(COP(=O)(O)O[C@H]2[C@H]1F)OC(n1cnc2c(=O)[nH]cnc21)[C@@H]3F] |
| CL647 | (2′,3′)c-(AMP- 2′FdIMP) | 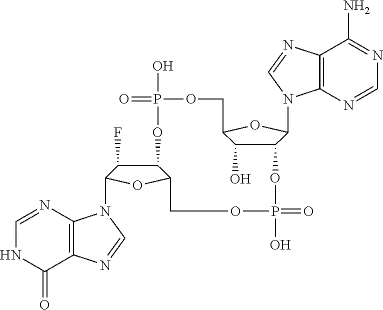 [SMILES: Nc1ncnc2c1ncn2C1OC2COP(=O)(O)O[C@@H]3C(COP(=O)(O)O[C@@H]1[C@@H]2O)OC(n1cnc2c(=O)[nH]cnc21)[C@@H]3F] |
| CL656 | c[2′FdAMP(S)- 2′FdIMP(S)] | 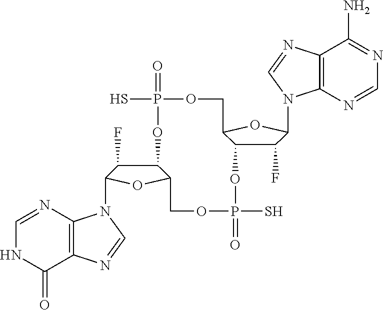 [SMILES: Nc1ncnc2c1ncn2C1OC2COP(=O)(S)O[C@@H]3C(COP(=O)(S)O[C@H]2[C@H]1F)OC(n1cnc2c(=O)[nH]cnc21)[C@@H]3F] |
| CL659 | c-[2′FdAMP(S)- 2′FdIMP(S)](POM)₂ | 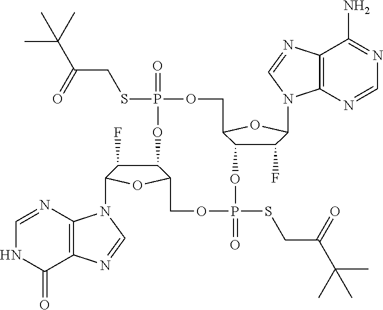 [SMILES: CC(C)(C)C(=O)CSP1(=O)OCC2OC(n3cnc4c(=O)[nH]cnc43)[C@H](F)[C@@H]2OP(=O)(SCC(=O)C(C)(C)C)OCC2OC(n3cnc4c(N)ncnc43)[C@H](F)[C@@H]2O1] |

These compounds can be produced by any method known by the skilled person in the art. For example, suitable methods for producing these compounds are described in the co-pending application PCT/EP2015/070635.

Thus, in a further embodiment, the present invention relates to a kit of parts comprising:
a. gemcitabine or a pharmaceutically acceptable salt or prodrug thereof; and
b. a cyclic dinucleotide or pharmaceutically acceptable salt or prodrug thereof, wherein said cyclic dinucleotide is selected from the group consisting of: c-AIMP, (3′,2′)c-AIMP, (2′,2′)c-AIMP, (2′,3′)c-AIMP, c-AIMP(S), c-(dAMP-dIMP), c-(dAMP-2′FdIMP), c-(2′FdAMP-2′FdIMP), (2′,3′)c-(AMP-2′FdIMP), c-[2′FdAMP(S)-2′FdIMP(S)] and c-[2′FdAMP(S)-2′FdIMP(S)](POM)₂ or a pharmaceutically acceptable salt or prodrug thereof for use in the treatment of solid pancreatic tumors.

Particularly, the cyclic dinucleotide is selected from the group consisting of: c-AIMP, c-(2′FdAMP-2′FdIMP), c-AIMP(S), c-[2′FdAMP(S)-2′FdIMP(S)] and c-[2′FdAMP(S)-2′FdIMP(S)](POM)₂.

The chemoimmunotherapy according to the invention provides greater treatment efficacy in three different animal models of pancreatic tumors than does gemcitabine monotherapy.

The specific combination of a chemotherapeutic agent with a cyclic dinucleotide or a pharmaceutically acceptable salt or prodrug thereof provides an efficient treatment for cancer, particularly pancreatic cancer.

The chemotherapeutic agent and the cyclic dinucleotide cooperate so as to provide a synergic effect between the two compounds.

The cyclic dinucleotides encompassed by the present invention offer several therapeutic and practical advantages for clinical use as immunotherapeutic agents. All the compounds presented in Table 1 are c-AIMP and c-AIMP analogs, including c-AIMP prodrugs. The other ten cyclic dinucleotides (c-AIMP analogs) disclosed in Table 1 possess equal or better STING agonist activity than that of c-AIMP.

Cyclic dinucleotides do not resemble typical small-molecule drug candidates: their molecular weight is ˜700 Da, they have two negative charges, and they are built from potentially labile phosphodiester linkages. Nevertheless, they are able to activate the STING pathway, presumably after entering the cell by presently unknown mechanisms. Unlike in many of the previously cited reports on cyclic dinucleotides (see, for example: (Ablasser et al., 2013) (Downey, Aghaei, Schwendener, & Jirik, 2014) and (Miyabe et al., 2014)), in which cells or animals are treated with a formulation comprising a cyclic dinucleotide and some type of complexing or transfection agent (e.g. liposomes), the cyclic dinucleotides according to the present invention can be administered to a subject without any kind of complexing or transfection agent. Moreover, there is no need to permeabilize cultured recipient cells (e.g. by using compounds such as digitonine) to favor uptake of CDNs. Indeed, in all of the in vitro and in vivo experiments supporting the present invention (see Examples 1 to 6), the cyclic dinucleotides were tested without the use of any complexing or transfection agent.

Since STING is located in the endoplasmic reticulum and detects cyclic dinucleotides in the cytoplasm, any STING agonist destined for therapeutic use must be able to penetrate into cells. Furthermore, greater cellular uptake of a compound translates to higher bioavailability, which is a desirable property for clinical use. We chose the fluorinated compounds CL609, CL614, CL647, CL656 and CL659 to explore the possibility that the greater cellular uptake conferred by one fluorine atom (in CL609 and CL647) or two fluorine atoms (in CL614, CL656 and CL659) would lead to greater Type I interferon induction activity than that of the reference compound, c-AIMP, which does not contain any fluorine atoms.

Cyclic dinucleotides are enzymatically degraded by nucleases and/or phosphodiesterases (see, for example: (Li et al., 2014) (Diner et al., 2013) (Danilchanka & Mekalanos, 2013) (Shanahan, Gaffney, Jones, & Strobel, 2013) (Simm, Morr, Kader, Nimtz, & Romling, 2004)) and therefore, when used as therapeutic agents, these compounds can suffer from diminished half-life. Advantageously the compounds CL655 and CL656 enable maximal half-life, and possibly higher activity, in vivo, as they contain phosphorothioate (also known as “P(S)” or “thiophosphate”) internucleotide linkages. The use of such linkages is a known strategy to circumvent enzymatic hydrolysis (see, for example: US 2014/0205653 A1). The phosphorothioate linkage introduces an additional chiral center on the phosphorus atom, which yields a diastereoisomer pair ([Rp] and [Sp]) at each phosphorothioate linkage. In the present invention, CL655, CL656 and CL659 were obtained and tested as racemic mixtures.

According to the present invention, the chemotherapeutic agent and the CDN may be administered as a pharmaceutical formulation(s) in a therapeutically effective amount by any of the accepted modes of administration, preferably by intravenous or intratumoral route.

### BRIEF DESCRIPTION OF THE FIGURES

FIG. 1. STING signaling in the cell. Activation of STING by cyclic dinucleotides (CDN) leads to activation of the IRF3 and NF-κB pathways and consequently, to induction of Type I interferons and of pro-inflammatory cytokines, respectively.

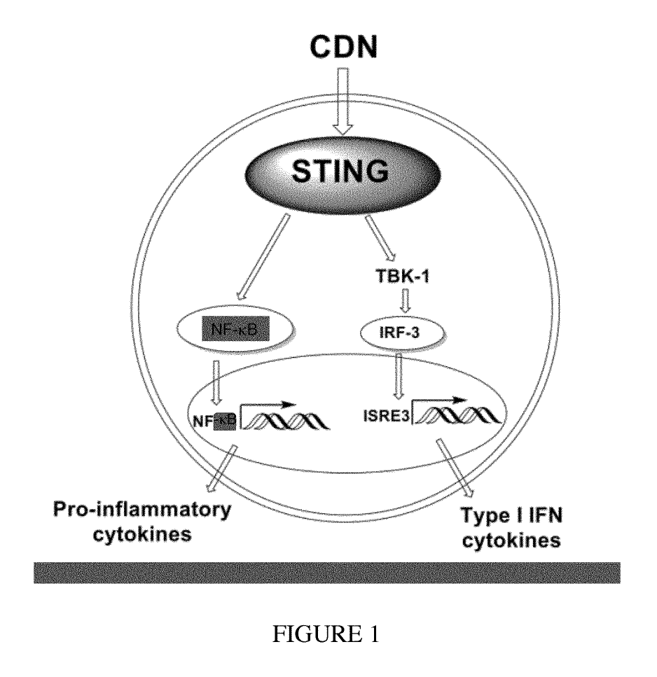

FIG. 2. In vitro Type I interferon induction activity in THP1-Dual™ cells. Values measured after 24 h incubation of the cyclic dinucleotides with the cells.

FIG. 3. In vitro Type I interferon induction activity in wild-type vs. STING knockout B16 cells. Relative ISG54 activity (as an indirect measurement of Type I interferon induction) of cyclic dinucleotides incubated with cultures of wild-type (right-side of graph) or STING-knockout (left-side of graph) B16 cells for 24 h. WT: wild-type; SKO: STING knockout (homozygous).

FIG. 4. In vitro Type I interferon induction activity in wild-type vs. STING-knockout RAW cells. Relative ISG54 activity (as an indirect measurement of Type I interferon induction) of cyclic dinucleotides incubated in cultures of wild-type (right-side of graph) or STING-knockout (left-side of graph) RAW cell for 24 h. WT: wild-type; SKO: STING knockout (homozygous).

FIG. 5. Type I interferon induction activity of cyclic dinucleotides in mice. Measurement of Type I interferon induction in sera from mice at 4 h post-treatment.

FIG. 6. IL-6 induction activity of cyclic dinucleotides in mice. Measurement of IL-6 induction in sera from mice at 4 h post-treatment.

FIG. 7. Tumor-growth inhibition in a murine model of Panc02 tumors. The mice were treated with saline (control), gemcitabine monotherapy, c-AIMP monotherapy, or gemcitabine combined with c-AIMP. *The Data for Day 28 are shown only for Group 1, as all the mice in this group had died by that day. GemC: gemcitabine; i.t.: intratumoral; i.v.: intravenous.

FIG. 8. Mean tumor volume in a hamster model of orthotopic PC-1.0 tumors (on Day 22). The hamsters were treated with saline, gemcitabine monotherapy, or gemcitabine combined with c-AIMP. Tumor volume was measured at the end of the experiment. GemC: gemcitabine; i.t.: intratumoral; i.v.: intravenous.

FIG. 9. Survival rate in a hamster model of orthotopic PC-1.0 tumors. The hamsters were treated with saline, gemcitabine monotherapy, or a combination of c-AIMP and gemcitabine. GemC: gemcitabine; i.t.: intratumoral; i.v.: intravenous.

FIG. 10. Tumor growth inhibition in the right-flank tumor in a hamster model of bilateral PC-1.0 tumors. The hamsters were treated in the right-flank tumor with saline, gemcitabine monotherapy, c-AIMP monotherapy, or gemcitabine combined with c-AIMP. GemC: gemcitabine.

FIG. 11. Tumor growth in mice implanted with orthotopic DT6606 pancreatic tumors. Pancreatic tumor (DT6606) growth at Day 36 post-implantation in mice treated with either gemcitabine (GemC) or an intercalated combination of CL592 and gemcitabine (CL592+GemC).

FIG. 12. Mean tumor volume in mice implanted with orthotopic Panc02 pancreatic tumors. Average tumor volume at Day 30 post-implantation was calculated for each group. Gem: gemcitabine.

### EXAMPLES

### Biological Assays

Before investigating the combination of gemcitabine with any of the cyclic dinucleotides encompassed by the present invention, the immunomodulatory activity of these cyclic dinucleotides was ascertained when used alone. These compounds induced the production of multiple cytokines in live human or animal cells. Specifically, these cyclic dinucleotides induce the production of Type I interferons and/or pro-inflammatory cytokines. The in vitro cytokine-induction activity of a representative set of these cyclic dinucleotides is reported here to require the presence of the eukaryotic cellular receptor known as “stimulator of interferon genes” (STING).

### In Vitro Cytokine Induction

The cytokine-induction activities of the cyclic dinucleotides disclosed in Table 1 have been demonstrated by using different reporter cell lines. The cell lines and experiments are explained below.

### Cell Lines

All the cell lines were obtained from InvivoGen. They are described here and provided with their corresponding InvivoGen catalog code.

THP1-Dual™ (Catalog Code: Thpd-Nfis):

These cells were derived from the human monocytic cell line THP-1 by stable integration of two inducible reporter constructs. They enable simultaneous study of the two main signaling pathways for STING: the NF-κB pathway, by monitoring the activity of secreted embryonic alkaline phosphatase (SEAP); and the IRF pathway, by assessing the activity of a secreted luciferase (Lucia).

Both reporter proteins are readily measurable in the cell culture supernatant when using QUANTI-Blue™ (InvivoGen catalog code: rep-qb1), a SEAP detection reagent that turns purple/blue in the presence of SEAP (quantified by measuring the optical density from 620 nm to 655 nm), and QUANTI-Luc™ (InvivoGen; catalog code: rep-q1c1), a luminometric enzyme assay that measures luciferase expression to report on ISG54 expression (as an indicator of IFN-α/β production).

Lucia ISG Cell Lines:

Each of the following three cell lines expresses a secreted luciferase (Lucia) reporter gene under control of an IRF-inducible promoter. This composite promoter comprises five IFN-stimulated response elements (ISREs) fused to a minimal promoter of the human ISG54 gene, which is unresponsive to activators of the NF-kB or AP-1 pathways. Hence, these cells enable monitoring of the IRF pathway based on luciferase (Lucia) activity.

In the present invention, monitoring of the IRF pathway is used to measure the STING agonist activity of the subject cyclic dinucleotides.
1. RAW-Lucia™ ISG (catalog code: rawl-isg): These cells were generated from the murine RAW 264.7 macrophage cell line.
2. RAW-Lucia™ ISG-KO-STING (catalog code: rawl-kostg): These cells were generated from the RAW-Lucia™ ISG54 cell line (see above), through stable homozygous knockout of the STING gene.

Blue™ Cell Lines:

Each of the following three cell lines expresses a SEAP reporter gene under a promoter: either I-ISG54, which comprises the IFN-inducible ISG54 promoter enhanced by a multimeric ISRE; or the IFN-β minimal promoter fused to five NF-κB (and five AP-1) binding sites. Stimulation of these cells with interferons, or inducers of type I interferons or of the NF-κB pathway, triggers activation of the I-ISG54 promoter (and consequently, production of SEAP) or of the IFN-β minimal promoter (and consequently, production of TNF-α). The levels of SEAP in the supernatant can be easily determined using QUANTI-Blue™ (InvivoGen catalog code: rep-qb1), a reagent that turns purple/blue in the presence of SEAP, by measuring the optical density from 620 nm to 655 nm.
1. B16-Blue™ ISG (catalog code: bb-ilhabg): These cells are derived from the murine B16 F1 melanoma cell line. Production of Type I interferons in these cells is measured using QUANTI-Blue™.
2. B16-Blue™ ISG-KO-STING (catalog code: bb-kostg): These cells were generated from the B16-Blue™ ISG cell line (see above), through stable homozygous knockout of the STING gene. Production of Type I interferons in these cells is measured using QUANTI-Blue™.

### Quantification of IL-6 in Experiments

Interleukin-6 was quantified using an enzyme-linked immunoassay (ELISA) according to the manufacturer's instructions (R&D Systems).

### In Cell Cultures

In various experiments in which different cell cultures were separately incubated with a cyclic dinucleotide, the cyclic dinucleotide induced production of Type I interferons and/or pro-inflammatory cytokines in those cells, as indirectly determined by an ISG54 (interferon-stimulated gene) reporter assay (Fensterl, White, Yamashita, & Sen, 2008). These experiments were performed as described below.

### Example 1: Measuring Cytokine Induction in Treated Cell Cultures

Cytokine reporter cell lines used: THP1-Dual™
Cyclic dinucleotides tested: CL602, CL604, CL606, CL609, CL611, CL614, CL647, CL655, CL656 and CL659
Reference compound: c-AIMP
Cytokines evaluated: IFN-α/β

To each well of a flat-bottom 96-well plate were added 20 μL of a solution a cyclic dinucleotide (100 μg/mL in sterile water), followed by 180 μL of a suspension of a single cell line (THP1-Dual™: ca. 100,000 cells per well). The plate was incubated for 18 h to 24 h at 37° C. in 5% CO₂. The level of IFN-α/β in each well was indirectly quantified using QUANTI-Luc™ (as an indicator of IFN-β production), which was prepared and used according to the manufacturer's instructions (InvivoGen).

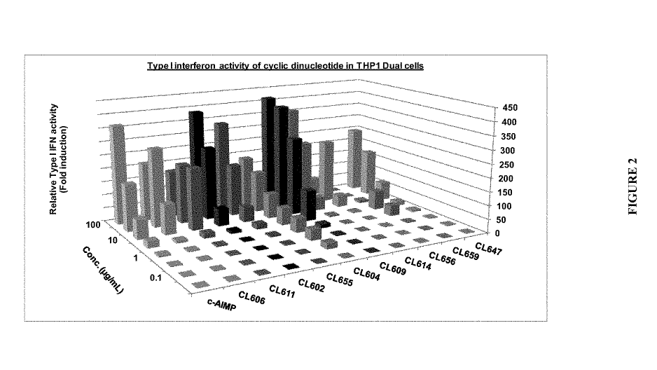

The results from this experiment are shown in FIG. 2, which illustrates that each one of the tested cyclic dinucleotides induces production of Type I interferons in THP1 cells.

### Cytokine Induction Activity is STING-Dependent

The cyclic dinucleotides disclosed in the present invention do not induce cytokine production in vitro in the supernatant of cells that lack the receptor STING.

In an experiment in which wild-type (WT) reporter cells and homozygous STING knockout (SKO) reporter cells were each separately incubated with the cyclic dinucleotide for 18 h to 24 h, the cyclic dinucleotide induced production of Type I interferons in the WT cells but not in the STING KO cells. This finding demonstrated that STING is required for the cytokine-induction activity of the cyclic dinucleotide in vitro in cells. These experiments were performed as described below:

### Example 2: Measuring Cytokine Induction in CDN-Treated Wild-Type or STING Knockout Cells

Cyclic dinucleotides tested: CL604, CL609, CL614, CL647, CL655 and CL656
Reference compounds: c-AIMP
Cytokines evaluated: IFN-α/β
Cell lines used: RAW-Lucia™ ISG, RAW-Lucia™ ISG-KO-STING, B16-Blue™ ISG, and B16-Blue™ ISG-KO-STING (depending on experiment)

To each well of a flat-bottom 96-well plate were added 20 μL of a solution a cyclic dinucleotide (100 μg/mL in sterile water), followed by 180 μL of a suspension of a single cell line (RAW-Lucia™ ISG: ca. 100,000 cells per well; B16-Blue™ ISG: ca. 50,000 cells per well). The plate was incubated for 18 h to 24 h at 37° C. in 5% CO₂. For the RAW cell lines, the level of IFN-α/β in each well was indirectly quantified using QUANTI-Luc™ (as an indicator of IFN-β production), which was prepared and used according to the manufacturer's instructions. For the B16 cell lines, the level of IFN-α/β in each well was indirectly quantified using QUANTI-Blue™, as described above.

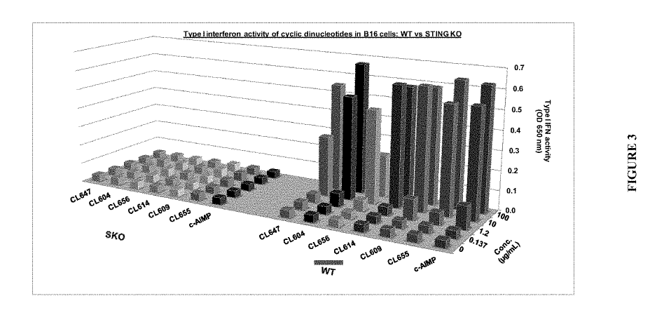

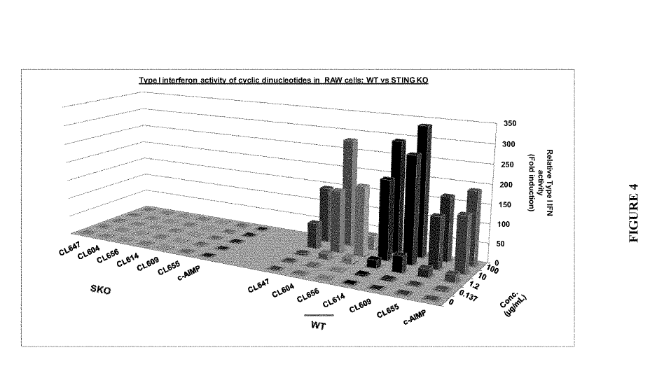

The results from this experiment are shown in FIGS. 3 and 4, which reveal three important findings. Firstly, each one of the tested cyclic dinucleotides induces production of Type I interferons in WT B16 (FIG. 3) and WT RAW (FIG. 4) cells. Secondly, none of the compounds exhibits this activity in STING knockout B16 (FIG. 3) or STING knockout RAW (FIG. 4) cells, thereby indicating that this activity requires the presence of STING. Lastly, the majority of the fluorinated cyclic dinucleotides are more active than is the reference compound (c-AIMP), as observed in the WT B16 (FIG. 3) and WT RAW (FIG. 4) cells.

### In Vivo Cytokine Induction

The cyclic dinucleotides disclosed in the present invention induce cytokines in vivo in mice.

### Example 3: Measuring Cytokine Induction in CDN-Treated Mice

Species evaluated: mouse
Cyclic dinucleotides tested: CL604, CL606, CL609, CL611 and CL614
Reference compound: c-AIMP and saline
Cytokines evaluated: IFN-α/β (using RAW ISG54 reporter cells) and IL-6 (by ELISA)

Twenty-one mice (Swiss; female; mean age: 8 weeks) were divided into seven groups of three: one group served as control (saline) and the other six groups were each treated with a cyclic dinucleotide (either c-AIMP, CL604, CL606, CL609, CL611 or CL614). On Day −7, blood samples for basal cytokine levels were collected from all mice and stored at −20° C. until analysis. On Day 1, the mice were treated with either 200 μL of physiologic serum (containing 0.9% NaCl) or 200 μL of a solution of a cyclic dinucleotide (dose: 10 mg/kg) in physiologic serum (containing 0.9% NaCl), by intravenous (i.v.) injection. Blood samples were collected from the mice at 4 h post-injection, and then stored at −20° C. until analysis. Cytokine induction was measured in the sera from the blood samples.

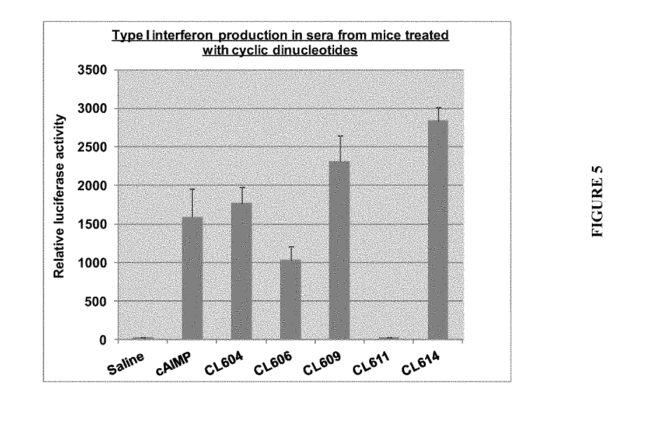

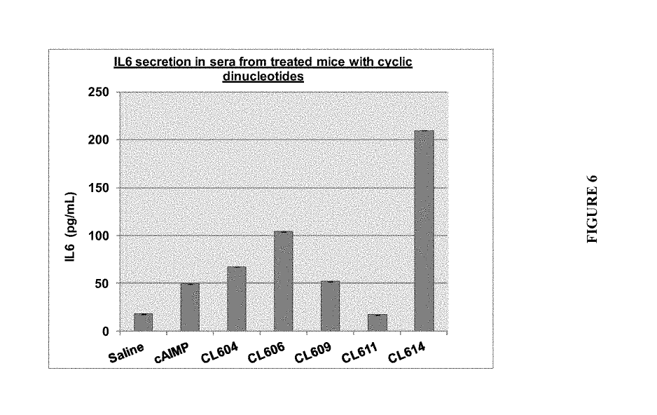

The results from this experiment are shown in FIGS. 5 and 6, which reveal two important findings: firstly, at the indicated dose, within 4 h post-treatment, all of the tested cyclic dinucleotides except CL611 strongly induced Type I interferons (FIG. 5) in mice; and secondly, all of the cyclic dinucleotides except CL611 induced IL-6 (FIG. 6).

In Vivo Efficacy of c-AIMP Combined with Gemcitabine

In experiments in which animal models of pancreatic cancer were treated with either gemcitabine monotherapy, c-AIMP monotherapy or chemoimmunotherapy (gemcitabine combined with c-AIMP), those animals that had received the combination therapy exhibited the greatest shrinkage in tumor volume, the lowest incidence of metastasis and/or the lowest mortality by the end of the experiment. Interestingly, in hamsters with bilateral subcutaneous pancreatic tumors, treatment of the right-flank tumor with chemoimmunotherapy (gemcitabine combined with c-AIMP) led to shrinkage of it as well as of the left-flank (distal) tumor.

The aforementioned experiments were performed as described below:

### Example 4: In Vivo Efficacy of Gemcitabine Combined with c-AIMP in a Murine Model of Pancreatic Cancer

Species evaluated: mouse
Tumor model: Panc02 (murine pancreatic tumor cell line)
Treatments tested: gemcitabine monotherapy, c-AIMP monotherapy, and gemcitabine combined with c-AIMP
Clinical parameters evaluated: tumor volume, incidence of metastasis and mortality
Administration routes evaluated: intravenous (i.v.) or intratumoral (i.t.) injection (depending on experiment)
On Day 1, 30 mice (C57BL/6; male) received an orthotopic injection of Panc02 tumor cells (1×10⁶) in their pancreas. The mice were then divided into six groups of five animals. Each group received a different treatment, as outlined below:
Group 1: saline (by i.v. injection) on Days 7, 10, 14, 17, 21 and 24;
Group 2: gemcitabine monotherapy (100 mg/kg; i.p.); on Days 7, 10, 14, 17, 21 and 24;
Group 3: c-AIMP monotherapy (25 mg/kg; i.t.) on Days 7 and 21;
Group 4: c-AIMP monotherapy (25 mg/kg; i.v.) on Days 7, 14 and 21;
Group 5: c-AIMP (25 mg/kg; i.t.) followed (5 h later) by gemcitabine (100 mg/kg; i.p.) on Day 7; and gemcitabine (100 mg/kg; i.p.) on Days 10, 14, 17, 21 and 24;
Group 6: c-AIMP (25 mg/kg; i.v.) followed (5 h later) by gemcitabine (100 mg/kg; i.p.) on Day 7; and gemcitabine (100 mg/kg; i.p.) on Days 10, 14, 17, 21 and 24;

At days 7, 21/24, 28 and 34, the mice were assessed for tumor volume, incidence of metastasis and mortality.

TABLE 2
Incidence of metastasis in a murine model of Panc02 tumors.
The mice were treated with saline (control), gemcitabine
monotherapy, c-AIMP monotherapy, or gemcitabine combined
with c-AIMP. All data from Day 34, except those for Group 1
(Day 28). GemC: gemcitabine; i.t.: intratumoral; i.v.: intravenous.

| | INCIDENCE OF |
|---|---|
| TREATMENT GROUP | METASTASIS |
| Group 1: Saline | 100% |
| Group 2: GemC | 50% |
| Group 3: cAIMP (i.t.) | 0% |
| Group 4: cAIMP (i.v.) | 0% |
| Group 5: cAIMP (i.t.) + GemC | 0% |
| Group 6: cAIMP (i.v.) + GemC | 40% |

TABLE 3
Mortality in a murine model of Panc02 tumors. The mice were
treated with saline (control), gemcitabine monotherapy, c-AIMP
monotherapy, or gemcitabine combined with c-
AIMP. All data from Day 34, except those for Group 1 (Day 28).

| | PRE-SACRIFICE |
|---|---|
| TREATMENT GROUP | MORTALITY |
| Group 1: Saline | 100% |
| Group 2: GemC | 20% |
| Group 3: cAIMP (i.t.) | 0% |
| Group 4: cAIMP (i.v.) | 0% |
| Group 5: cAIMP (i.t.) + GemC | 0% |
| Group 6: cAIMP (i.v.) + GemC | 0% |

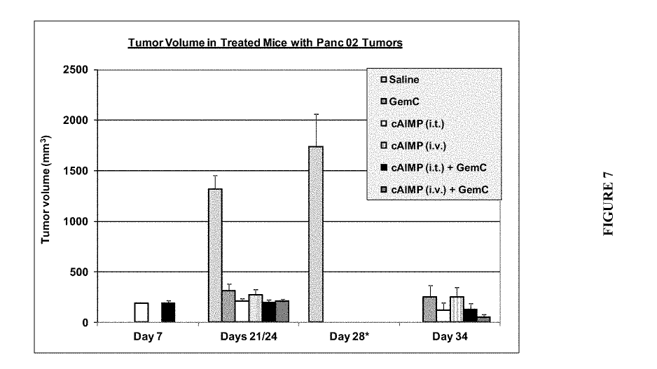

The results from this experiment are shown in FIG. 7 and in Tables 2 and 3. FIG. 7 reveals that among all of the treatments tested, the most effective ones at reducing tumor growth were c-AIMP monotherapy and the two combination treatments (gemcitabine plus c-AIMP [i.v. or i.t.]). Table 2 indicates that among the six treatment groups, the lowest incidences of metastasis were found in all four groups that had received c-AIMP (either alone or in combination with gemcitabine). Likewise, Table 3 shows that in these same four groups, the pre-sacrifice mortality rate by Day 34 was 0%, compared to 20% for the gemcitabine monotherapy group and 100% (by Day 28) for the saline group.

### Example 5: In Vivo Efficacy of c-AIMP Combined with Gemcitabine in a Hamster Model of Pancreatic Cancer (Orthotopic Tumor)

Species evaluated: hamster
Tumor model: PC-1.0 (hamster pancreatic tumor cell line (Egami, Tomioka, Tempero, Kay, & Pour, 1991))
Treatments tested: gemcitabine monotherapy, and gemcitabine combined with c-AIMP
Clinical parameters evaluated: tumor volume, incidence of metastasis and mortality
Administration routes evaluated: intravenous (i.v.) vs. intratumoral (i.t.) injection (depending on experiment) for CL592
On Day 1, 22 hamsters (Golden Syrian) received an orthotopic injection of PC-1.0 tumor cells (1×10⁶) in the tail of their pancreas. The hamsters were then divided into four groups of five or six animals. Each group received a different treatment, as outlined below:
Group 1 (n=5) received saline (by i.v. injection) on Days 8, 15 and 22;
Group 2 (n=6): c-AIMP (25 mg/Kg; i.v.) followed by gemcitabine (50 mg/Kg; i.p.) on Day 8; and gemcitabine (50 mg/Kg; i.p.) on Days 15 and 22;
Group 3 (n=6): c-AIMP (25 mg/Kg; i.t.) followed by gemcitabine (50 mg/Kg; i.p.) on Day 8; and gemcitabine (50 mg/Kg; i.p.) on Days 15 and 22;
Group 4 (n=5): gemcitabine monotherapy (50 mg/Kg; i.p.) at Days 8, 15 and 22.

At days 8, 21/24, 28 and 34, the mice were assessed for tumor volume, incidence of metastasis and mortality.

TABLE 4
Incidence of metastases in a hamster model of orthotopic
PC-1.0 tumors. The hamsters were treated with saline,
gemcitabine monotherapy, or a combination of c-AIMP
and gemcitabine. GemC: gemcitabine; i.t.: intratumoral;
i.v.: intravenous.

| | INCIDENCE |
|---|---|
| TREATMENT GROUP | OF METASTASES |
| Group 1: Saline | 100% |
| Group 2: cAIMP (i.v.) + GemC | 0% |
| Group 3: cAIMP (i.t.) + GemC | 0% |
| Group 4: GemC | 100% |

TABLE 5
Number of metastases in a hamster model of orthotopic PC-1.0 tumors.
The hamsters were treated with saline, gemcitabine monotherapy, or a
combination of c-AIMP and gemcitabine. GemC: gemcitabine;
i.t.: intratumoral; i.v.: intravenous.

| TREAT- | | | | | |
|---|---|---|---|---|---|
| MENT | NUMBER OF METASTASES PER HAMSTER | | | | |
| GROUP | Hamster 1 | Hamster 2 | Hamster 3 | Hamster 4 | Hamster 5 |
| Group 1: | 28 | 10 | 17 | 3 | 20 |
| Saline | | | | | |
| Group 2: | 0 | 0 | 0 | 0 | 0 |
| cAIMP | | | | | |
| (i.v.) + | | | | | |
| GemC | | | | | |
| Group 3: | 0 | 0 | 0 | 0 | 0 |
| cAIMP | | | | | |
| (i.t.) + | | | | | |
| GemC | | | | | |
| Group 4: | 13 | 15 | 3 | 10 | 5 |
| GemC | | | | | |

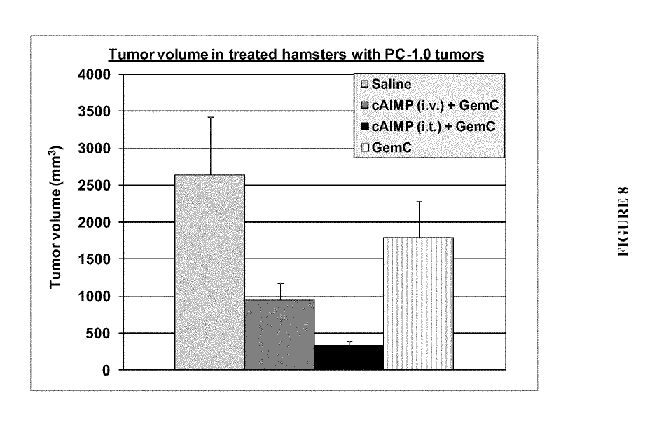

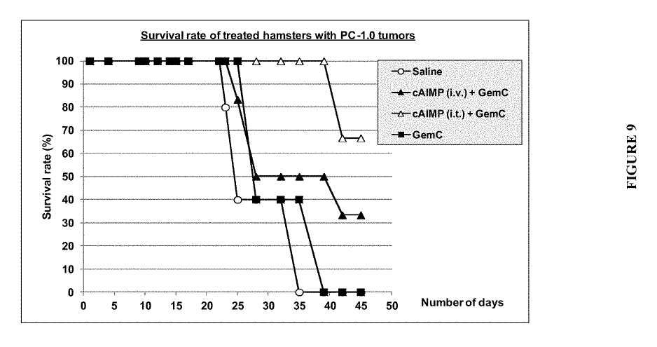

The results from this experiment are shown in FIGS. 8 and 9, and in Tables 4 and 5. FIG. 8 reveals that among the four treatments tested, both combination therapies were better at reducing tumor growth than was gemcitabine monotherapy, and that the better of the combination therapies was gemcitabine plus c-AIMP (i.t.). Similarly, FIG. 9 illustrates that gemcitabine plus c-AIMP (i.t.) provided the highest survival rate. Table 4 shows that none (0% incidence) of the hamsters in the two combination-treatment groups exhibited any metastases, whereas all (100% incidence) of the hamsters in both the gemcitabine monotherapy group and the saline group exhibited metastases. Table 5 lists the number of metastases per hamster in each group, showing a value of zero for every hamster in the two combination-treatment groups.

### Example 6: In Vivo Efficacy of c-AIMP Combined with Gemcitabine in a Hamster Model of Subcutaneous Pancreatic Tumors (Bilateral)

Species evaluated: hamster
Tumor model: PC-1.0 (see above)
Treatments tested: gemcitabine monotherapy, c-AIMP monotherapy, and gemcitabine combined with c-AIMP
Clinical parameters evaluated: tumor volume at right (treated) flank, incidence of metastasis and mortality
Administration routes evaluated: intravenous (i.v.) or intratumoral (i.t.) injection (depending on experiment)
On Day 1, 25 hamsters (Golden Syrian) received a subcutaneous injection of PC-1.0 cells (1×10⁶) in the right flank. On Day 6, the hamsters received a subcutaneous injection of PC-1.0 cells (1×10⁵) in their left flank. On Day 7, 25 of the hamsters were randomly assigned (based on right-flank tumor size) to groups of five animals each. Each group received a different treatment, as outlined below:
Group 1: saline (i.t.) on Day 8;
Group 2: saline (i.t.) followed (3 h later) by gemcitabine (50 mg/kg; i.p.) in saline on Day 8; gemcitabine (50 mg/kg; i.p.) in saline on Days 15 and 22;
Group 3: c-AIMP (25 mg/kg; intratumoral injection in right-flank tumor) on Day 8 and, if a tumor was present, on Day 22;
Group 4: c-AIMP (25 mg/kg; intratumoral injection in right-flank tumor) followed (3 h later) by gemcitabine (50 mg/kg; i.p.) on Day 8; gemcitabine (50 mg/kg; i.p.) on Day 15; if a tumor was present, c-AIMP (25 mg/kg; intratumoral injection in right-flank tumor) on Day 22 and in all cases, gemcitabine (50 mg/kg; i.p.) on Days 15 and 22.

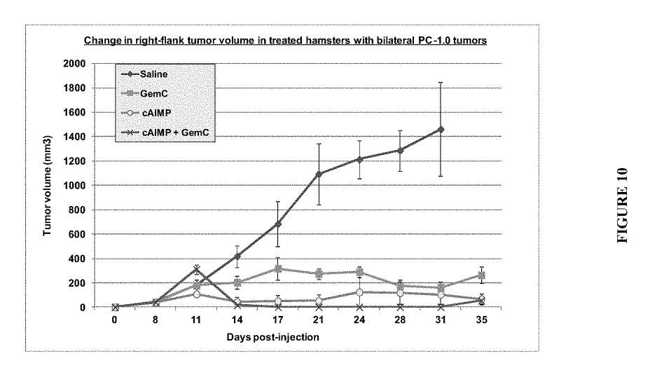

The results from this experiment are shown in FIG. 10, which reveals that over the course of the experiment, the most effective treatment at reducing tumor growth was the combination of gemcitabine and c-AIMP. In fact, the hamsters treated with this combination treatment exhibited the smallest tumor volume at all time points measured except for one (Day 11 post-injection).

### Example 7: Comparison of Gemcitabine with an Intercalated Combination of CL592 and Gemcitabine in an Orthotopic Murine Model of Pancreatic Cancer

Tumor line evaluated: DT6606 (Partecke, 2011)
Treatment tested: intercalated combination of CL592 and gemcitabine
Reference compound: gemcitabine
Parameter evaluated: tumor growth

On Day 1, 20 mice (C57/BL6; female; 10 weeks old; 18 g to 22 g) received an intrapancreatic injection of DT6606 cells (5×10⁵ cells in 30 μL serum-free medium). One mouse was sacrificed before treatment due to a renal deformation. The remaining mice were divided into four groups (n=5, except for Group 2: n=4), as shown in the table below:

| Group | Treatment |
|---|---|
| 1 | saline (control) |
| 2 | gemcitabine |
| 3 | CL 592 |
| 4 | CL592 + gemcitabine |

On Day 13, tumor growth was confirmed in all the mice and the volume of each tumor was measured. The groups were then treated according to the treatment regimen below.

### Treatment Regimen

Day 13: Groups 1 and 2 received an intratumoral injection of saline solution (50 μL), and Groups 3 and 4, an intratumoral injection of CL592 (50 μL; 2.5 mg/mL in saline buffer;).

Day 16: Groups 2 and 4 received an intraperitoneal tail injection of gemcitabine (100 μL solution/100 g body mass; 10 mg/mL in saline buffer; dose: 100 mg/kg).

Day 20: Groups 3 and 4 received an intravenous tail injection of CL592 (200 μL; 0.5 mg/mL in saline buffer; dose: 5 mg/kg).

Day 23: Groups 2 and 4 were treated as on Day 16.

Day 27: Groups 3 and 4 were treated as on Day 20.

Day 30: Groups 2 and 4 were treated as on Days 16 and 23.

Day 36: Each mouse was checked for tumor presence. The volume of each observed tumor was measured and the mice were then sacrificed.

Tumor growth (expressed as a percentage) was calculated as follows:

(([tumor volume at day 36]−[pre-treatment tumor volume])/[pre-treatment tumor volume])×100%

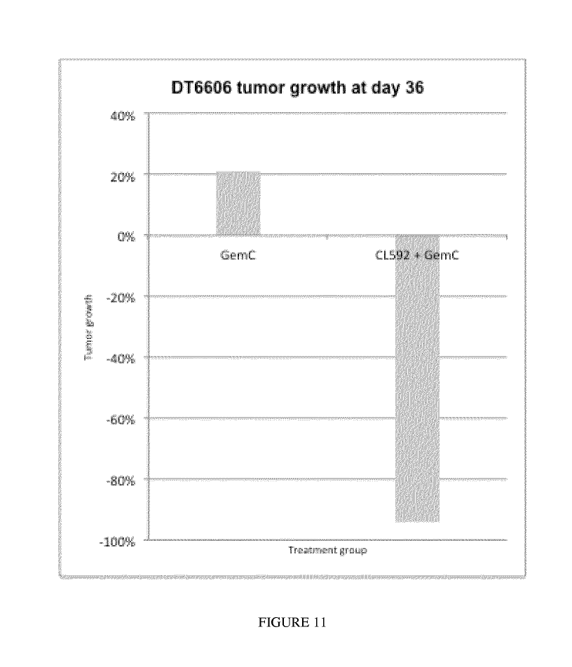

The principal result from this experiment is shown in FIG. 11, which reveals that the intercalated combination of CL592 and gemcitabine was markedly more effective at stopping tumor growth than was gemcitabine monotherapy. Specifically, by the end of the experiment (Day 36), the tumors in the combination group had shrunk drastically (mean growth: −94%), whereas those in the gemcitabine group had actually grown slightly (mean growth: 22%).

### Example 8: Evaluation of Different Intercalated Combinations of a CDN and Gemcitabine in an Orthotopic Murine Model of Pancreatic Cancer

Species evaluated: mouse
Tumor model: Panc02
Treatment tested: intercalated combinations of a CDN (either CL592, CL614 or CL656) and gemcitabine
Reference compounds: gemcitabine, CL592, CL614 and CL656
Parameters evaluated: tumor growth, and incidence of metastases

On Day 1, 55 mice (C57/BL6; male; 10 weeks old; 23 g to 25 g) each received an intrapancreatic injection of Panc02 cells (1×10⁶ cells in 50 μL serum-free medium). The mice were divided into eight groups, as shown in the table below:

| | | Number |
|---|---|---|
| Group | Treatment | of mice |
| 1 | saline (control) | 8 |
| 2 | gemcitabine | 8 |
| 3 | CL592 | 5 |
| 4 | CL614 | 5 |
| 5 | CL656 | 5 |
| 6 | CL592 + gemcitabine | 8 |
| 7 | CL614 + gemcitabine | 8 |
| 8 | CL656 + gemcitabine | 8 |

The groups were treated according to the treatment regimen below.

### Treatment Regimen

Day 9: Groups 3 to 8 each received an intratumoral injection of the appropriate CDN (CL592, CL614 or CL656, respectively; 50 μL; 5 mg/kg in 0.9% saline)

Day 12: Group 2 and Groups 6 to 8 each received an intraperitoneal injection of gemcitabine (200 μL; 100 mg/kg in 0.9% saline)

Day 16: Groups 3 to 8 each received an intravenous injection of the appropriate CDN (CL592, CL614 or CL656, respectively; 50 μL; 5 mg/kg in 0.9% saline)

Day 19: Group 2 and Groups 6 to 8 were treated as on Day 12.

Day 23: Groups 3 to 8 were treated as on Day 16.

Day 26: Group 2 and Groups 6 to 8 were treated as on Days 12 and 19.

Day 30: Each mouse was checked for tumor presence and metastases. The volume of each observed tumor was measured, any observed metastases were counted and then, the mice were sacrificed.

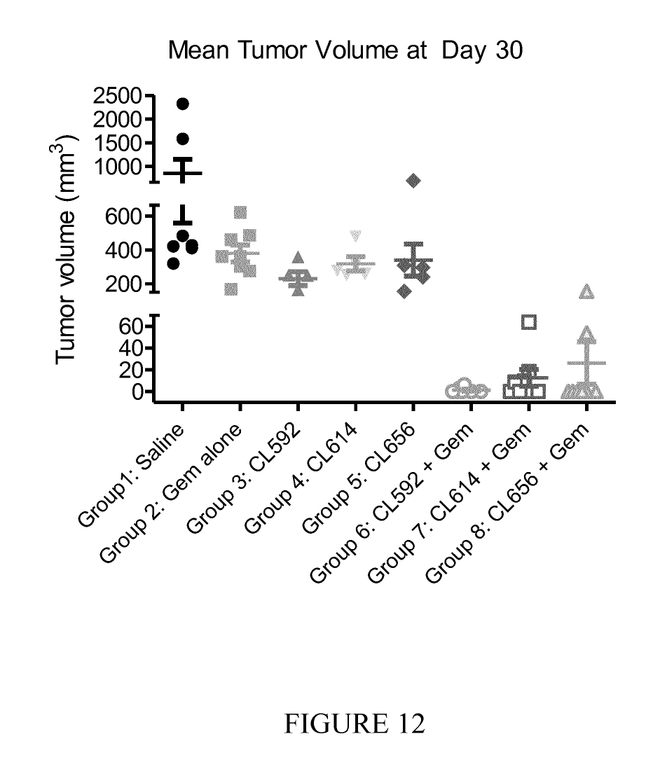

The principal results from this experiment are shown in FIG. 12 and Table 6, which reveal that the intercalated combination of any one of the CDNs and gemcitabine was markedly more effective at stopping tumor growth (FIG. 12) and preventing metastasis (Table 6) than was any of the tested single reference compounds (gemcitabine, CL592, CL614 or CL656). Specifically, by the end of the experiment (Day 30), the mean tumor volume in each combination group (Groups 3: 1.3 mm³±2.2 mm³; Group 4: 12.6 mm³±21.7 mm³; and Group 5: 26.1 mm³±55.3 mm³) was hundreds of times smaller than that of the gemcitabine group (380.7 mm³±140.9 mm³), the CL592 group (231.0 mm³±90.0 mm³), the CL614 group (318.6 mm³±93.8 mm³), the CL656 group (340.2 mm³±210. mm³) or the saline group (854.4 mm³±784.1 mm³).

Interestingly, the results from this and another experiment on Panc02 in mice provide important insight on the dosage of CL592 to be used: at lower doses (5 mg/kg; Example 8), the combination of gemcitabine and CL592 provides a clear beneficial effect relative to either component alone, whereas at a far higher dose (25 mg/kg; Example 4), this effect is less pronounced. This observation could ultimately have crucial implications for development of a clinical treatment regimen based on our proposed combination of gemcitabine and a CDN STING agonist: for example, in trying to maximize the efficacy of the combination while minimizing the respective toxicity of each component.

TABLE 6
Incidence of metastases in mice implanted with orthotopic
Panc02 pancreatic tumors. The number of animals with
metastasis and the average number of metastases per
animal at Day 30 post-implantation were calculated for
each group. Note that one of the mice in Group 1 had
died before Day 30.

| | | # Mice with | Average # metastases |
|---|---|---|---|
| Group | Treatment | metastases | per mouse |
| 1 | saline (control) | 100% (7/7) | 17 |
| 2 | gemcitabine | 88% (7/8) | 13 |
| 3 | CL592 | 20% (1/5) | 2 |
| 4 | CL614 | 40% (2/5) | 5 |
| 5 | CL656 | 40% (2/5) | 3 |
| 6 | CL592 + gemcitabine | 0% (0/8) | 0 |
| 7 | CL614 + gemcitabine | 0% (0/8) | 0 |
| 8 | CL656 + gemcitabine | 0% (0/8) | 0 |

### REFERENCES

1. Abdul-Sater, A. A. et al. Cyclic-di-GMP and cyclic-di-AMP activate the NLRP3 inflammasome. EMBO Rep 14, 900-6 (2013).
2. Ablasser, A. et al. cGAS produces a 2′-5′-linked cyclic dinucleotide second messenger that activates STING. Nature 498, 380-4 (2013).
3. Bundgaard, H. Design of prodrugs (Elsevier; Sole distributors for the USA and Canada, Elsevier Science Pub. Co., Amsterdam; New York; New York, N.Y., USA, 1985).
4. Chandra, D. et al. STING ligand c-di-GMP improves cancer vaccination against metastatic breast cancer. Cancer Immunol Res 2, 901-10 (2014).
5. Danilchanka, O. & Mekalanos, J. J. Cyclic dinucleotides and the innate immune response. Cell 154, 962-70 (2013).
6. Diner, E. J. et al. The innate immune DNA sensor cGAS produces a noncanonical cyclic dinucleotide that activates human STING. Cell Rep 3, 1355-61 (2013).
7. Downey, C. M., Aghaei, M., Schwendener, R. A. & Jirik, F. R. DMXAA causes tumor site-specific vascular disruption in murine non-small cell lung cancer, and like the endogenous non-canonical cyclic dinucleotide STING agonist, 2′3′-cGAMP, induces M2 macrophage repolarization. PLoS One 9, e99988 (2014).
8. Dubensky, T. W., Jr., Kanne, D. B. & Leong, M. L. Rationale, progress and development of vaccines utilizing STING-activating cyclic dinucleotide adjuvants. Ther Adv Vaccines 1, 131-43 (2013).
9. Fensterl, V., White, C. L., Yamashita, M. & Sen, G. C. Novel characteristics of the function and induction of murine p56 family proteins. J Virol 82, 11045-53 (2008).
10. Foundation, H. (2014).
11. Fritz, J. et al. In vitro immunomodulatory properties of gemcitabine alone and in combination with interferon-alpha. Immunol Lett 168, 111-9 (2015).
12. Gomelsky, M. cAMP, c-di-GMP, c-di-AMP and now cGMP: bacteria use them all! Mol Microbiol 79, 562-5 (2011).
13. Hirooka, Y. et al. A combination therapy of gemcitabine with immunotherapy for patients with inoperable locally advanced pancreatic cancer. Pancreas 38, e69-74 (2009).
14. Ishikawa, H., Ma, Z. & Barber, G. N. STING regulates intracellular DNA-mediated, type I interferon-dependent innate immunity. Nature 461, 788-92 (2009).
15. Karaolis, D. K. et al. 3′,5′-Cyclic diguanylic acid (c-di-GMP) inhibits basal and growth factor-stimulated human colon cancer cell proliferation. Biochem Biophys Res Commun 329, 40-5 (2005).
16. Kimura, Y. et al. Clinical and immunologic evaluation of dendritic cell-based immunotherapy in combination with gemcitabine and/or S-1 in patients with advanced pancreatic carcinoma. Pancreas 41, 195-205 (2012).
17. Klarquist, J. et al. STING-Mediated DNA Sensing Promotes Antitumor and Autoimmune Responses to Dying Cells. J Immunol (2014).
18. Li, L. et al. Hydrolysis of 2′3′-cGAMP by ENPP1 and design of nonhydrolyzable analogs. Nat Chem Biol 10, 1043-8 (2014).
19. Malvezzi, M., Bertuccio, P., Levi, F., La Vecchia, C. & Negri, E. European cancer mortality predictions for the year 2013. Ann Oncol 24, 792-800 (2013).
20. Mitzel, D. N., Lowry, V., Shirali, A. C., Liu, Y. & Stout-Delgado, H. W. Age-enhanced endoplasmic reticulum stress contributes to increased Atg9A inhibition of STING-mediated IFN-beta production during Streptococcus pneumoniae infection. J Immunol 192, 4273-83 (2014).
21. Miyabe, H. et al. A new adjuvant delivery system ‘cyclic di-GMP/YSK05 liposome’ for cancer immunotherapy. J Control Release 184, 20-7 (2014).
22. Murugesan, S. R. et al. Combination of human tumor necrosis factor-alpha (hTNF-alpha) gene delivery with gemcitabine is effective in models of pancreatic cancer. Cancer Gene Ther 16, 841-7 (2009).
23. Nishida, S. et al. Wilms tumor gene (WT1) peptide-based cancer vaccine combined with gemcitabine for patients with advanced pancreatic cancer. J Immunother 37, 105-14 (2014).
24. Ohkuri, T. et al. STING contributes to anti-glioma immunity via triggering type-I IFN signals in the tumor microenvironment. Cancer Immunol Res (2014).
25. Partecke, L. I. et al. A syngeneic orthotopic murine model of pancreatic adenocarcinoma in the C57/BL6 mouse using the Panc02 and 6606PDA cell lines. Eur Surg Res 47, 98-107 (2011).
26. Romling, U. & Amikam, D. Cyclic di-GMP as a second messenger. Curr Opin Microbiol 9, 218-28 (2006).
27. Shindo, Y. et al. Adoptive immunotherapy with MUC1-mRNA transfected dendritic cells and cytotoxic lymphocytes plus gemcitabine for unresectable pancreatic cancer. J Transl Med 12, 175 (2014).
28. Simm, R., Morr, M., Kader, A., Nimtz, M. & Romling, U. GGDEF and EAL domains inversely regulate cyclic di-GMP levels and transition from sessility to motility. Mol Microbiol 53, 1123-34 (2004).
29. Sloan, K. B. Prodrugs: topical and ocular drug delivery (M. Dekker, New York, 1992).
30. Society, A. C. (2014).
31. Suzuki, E., Kapoor, V., Jassar, A. S., Kaiser, L. R. & Albelda, S. M. Gemcitabine selectively eliminates splenic Gr-1+/CD11b+ myeloid suppressor cells in tumor-bearing animals and enhances antitumor immune activity. Clin Cancer Res 11, 6713-21 (2005).
32. Tomimaru, Y. et al. Synergistic antitumor effect of interferon-ss with gemcitabine in interferon-alpha-non-responsive pancreatic cancer cells. Int J Oncol 38, 1237-43 (2011).
33. Zhang, X. et al. Cyclic GMP-AMP containing mixed phosphodiester linkages is an endogenous high-affinity ligand for STING. Mol Cell 51, 226-35 (2013).

1-12. (canceled)

13. A method for treating a solid pancreatic tumor in a patient comprising administering to said patient a therapeutically effective amount of gemcitabine or a pharmaceutically acceptable salt thereof; and a therapeutically effective amount of a cyclic dinucleotide or pharmaceutically acceptable salt thereof,
said cyclic dinucleotide or pharmaceutically acceptable salt thereof being an agonist of the receptor known as “stimulator of interferon genes” (STING),
wherein said gemcitabine or a pharmaceutically acceptable salt thereof and said cyclic dinucleotide or a pharmaceutically acceptable salt thereof are administered to said patient in a separate form, either simultaneously or sequentially.

14. The method according to claim 13, wherein the nitrogenous base of each nucleoside of the cyclic dinucleotide is a purine that is substituted only in position 6.

15. The method according to claim 13, wherein one nucleoside of said cyclic dinucleotide is adenosine and the other nucleoside is inosine.

16. The method according to claim 13, wherein the linkage between the two nucleosides of said cyclic dinucleotide is a (3′,5′)(3′,5′), a (3′,5′)(2′,5′), a (2′,5′)(3′,5′) or a (2′,5′),(2′,5′) phosphodiester and/or phosphorothioate diester linkage.

17. The method according to claim 13, wherein said cyclic dinucleotide is represented by the following formula:

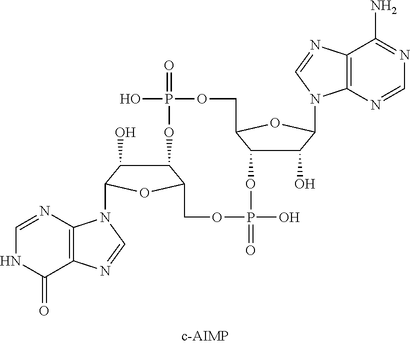 [SMILES: Nc1ncnc2c1ncn2C1OC2COP(=O)(O)O[C@@H]3C(COP(=O)(O)O[C@H]2[C@H]1O)OC(n1cnc2c(=O)[nH]cnc21)[C@@H]3O]

18. The method according to claim 13, wherein said cyclic dinucleotide is represented by the following formula:

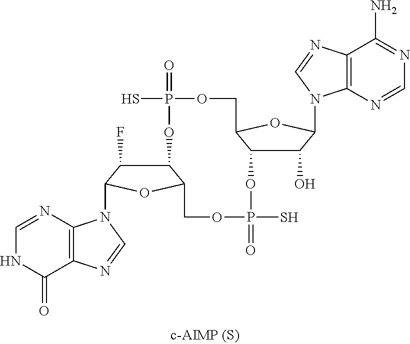 [SMILES: Nc1ncnc2c1ncn2C1OC2COP(=O)(S)O[C@@H]3C(COP(=O)(S)O[C@H]2[C@H]1O)OC(n1cnc2c(=O)[nH]cnc21)[C@@H]3F]

19. The method according to claim 13, wherein said cyclic dinucleotide is represented by the following formula:

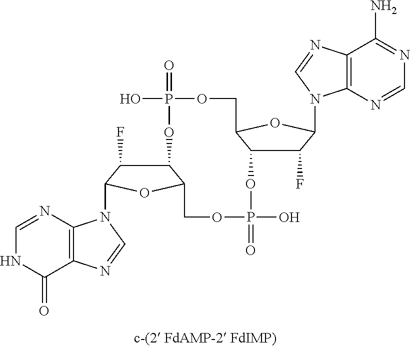 [SMILES: Nc1ncnc2c1ncn2C1OC2COP(=O)(O)O[C@@H]3C(COP(=O)(O)O[C@H]2[C@H]1F)OC(n1cnc2c(=O)[nH]cnc21)[C@@H]3F]

20. The method according to claim 13, wherein said cyclic dinucleotide is represented by the following formula:

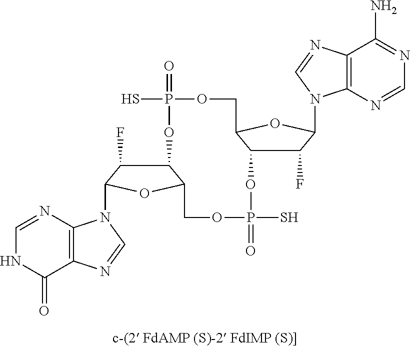 [SMILES: Nc1ncnc2c1ncn2C1OC2COP(=O)(S)O[C@@H]3C(COP(=O)(S)O[C@H]2[C@H]1F)OC(n1cnc2c(=O)[nH]cnc21)[C@@H]3F]

21. The method according to claim 13, wherein said cyclic dinucleotide is represented by the following formula:

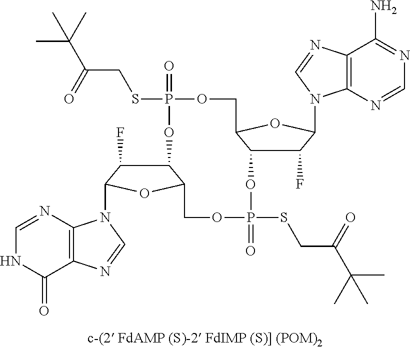 [SMILES: CC(C)(C)C(=O)CSP1(=O)OCC2OC(n3cnc4c(=O)[nH]cnc43)[C@H](F)[C@@H]2OP(=O)(SCC(=O)C(C)(C)C)OCC2OC(n3cnc4c(N)ncnc43)[C@H](F)[C@@H]2O1]

22. The method according to claim 13, wherein gemcitabine is administered by intravenous perfusion.

23. The method according to claim 13, wherein the cyclic dinucleotide is administered by intravenous perfusion or by intratumoral injection.
